(19) United States
(12) Patent Application Publication
(10) Pub. No.: US 2011/0020933 A1
(43) Pub. Date: Jan. 27, 2011

(54) METHOD AND DEVICE FOR RECEIVING BIOLOGICAL CELLS FROM A STEM CELL CULTURE

(75) Inventors: Charli Kruse, Herrnburg (DE); Guenter R. Fuhr, Berlin (DE)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: Fraunhofer-Gesellschaft zur Foerderung der angewandten Forschung e.V., Muenchen (DE)

(21) Appl. No.: 12/671,534

(22) PCT Filed: Jul. 25, 2008
(86) PCT No.: PCT/EP08/06141

§ 371 Date: Feb. 1, 2010

(30) Foreign Application Priority Data
Aug. 2, 2007 (DE) 10 2007 036 150.7

(51) Int. Cl. C12N 5/074

(52) U.S. Cl. 435/395

(57) ABSTRACT

A method for receiving biological cells (1, 2) from a cell culture (3) includes the steps: cultivation of the cell culture on a main substrate (10), cell transfer from the main substrate (10) onto at least one receiving substrate (20, 20.1, 20.2, 20.3, 20.4 . . . ), wherein the cells (1, 2) move onto the at least one receiving substrate (20, 20.1, 20.2, 20.3, 20.4 . . . ) by natural cell locomotion, and a cultivation of the cells (1, 2) is provided on the at least one receiving substrate (20, 20.1, 20.2, 20.3, . . . ), and separation of the main substrate (10) and of the at least one receiving substrate (20, 20.1, 20.2, 20.3, 20.4 . . . ) from one another. Also described is a substrate arrangement (100) for providing biological cells (1, 2) of a cell culture (3).

The invention relates to a method for receiving biological cells from a plurality of stem cells, in which the cells to be received migrate by their natural cell locomotion onto a receiving substrate. In addition, the invention relates to a substrate arrangement for receiving biological cells from a plurality of stem cells, wherein the substrate arrangement has two substrates, between which the cells to be received can be transferred by their natural cell locomotion.

It is known that adherent biological cells on a substrate perform natural cell locomotion. Cell locomotion generally comprises a change in position of a complete cell on the solid surface of the substrate or in cellular material by a rearrangement of adhesion contacts of cellular organs (membrane organs, for example membrane protrusions). Natural cell locomotion is described for example by M. Abercrombie et al. in “Experimental Cell Research”, Vol. 67, 1971, p. 359-367 and by L. P. Cramer in “Biochem. Soc. Symp.”, Vol. 65, 1999, p. 173-205.

A practical application of natural cell locomotion is the stress-free and damage-free removal of cells from in-vitro cultures. For example, US 2006/051735 A1 describes a transfer of biological cells by natural cell locomotion between a carrier and a probe. As transfer takes place by natural cell locomotion, in this method mechanical or biochemical effects on the cells are minimized and unphysiological interventions, for example trypsinization of the cellular material, are avoided.

The combination of a carrier and a probe described in US 2006/051735 A1 represents a special tool, whose practical usability may be restricted by the following problems. As the probe is intended for receiving an individual cell from the carrier or for transferring an individual cell onto the carrier, transfer of a plurality of cells requires serial operation or the use of a plurality of probes. In the first case there is the disadvantage that it takes a long time, whereas the second variant would be restricted for reasons of space to the combination of few probes. Moreover, the probe is only suitable for transport of the cell. For further investigations or treatments the cell must be transferred from the probe to another substrate.

It may also be a disadvantage that all cells participating in the transfer, i.e. both the cells in the cellular material on the carrier and the cell to be transferred have natural cell locomotion. The selective removal of individual cells or small groups of cells from the cellular material on the carrier therefore requires measures to prevent transfer of unwanted cells. It may for example be necessary to move the carrier and the probe during transfer. Moreover, for the selective removal of particular cells it may be necessary that the orientation of the carrier and of the probe and the migration of the cell in question are observed with a microscope. Owing to the aforementioned disadvantages, there are limitations with respect to automation of the conventional technology.

Another disadvantage of the conventional technology is that for selective transfer of particular cells, prior knowledge about these cells is required. If, for example in a stem cell culture in which some cells have differentiated (for example differentiation to nerve cells), the differentiated cells are to be removed for further investigation or use, the differentiated cells in the stem cell culture must be identified. Until now this has required an invasive intervention in the stem cell culture, for example specific staining, which represents an undesirable influence on the cells.

In in-vitro cultivation, biological cells on a substrate typically form a cell coating with a few cell layers or just one cell layer (monolayer). Certain cell types are distinguished in that they grow out beyond the thickness of the monolayer. For example, in the adherent state stem cells form three-dimensional cell aggregates (so-called organoid bodies, see C. Kruse et al. in “Appl. Phys. A”, Vol. 79, 2004, p. 1617-1624; and C. Kruse et al. in “Ann. Anat.”, Vol. 188 (6), 2006, p. 503-517). It is also known from these publications that stem cells or differentiated cells grow out of the cell aggregates, and also exhibit the aforementioned natural cell locomotion (see in addition S. Danner et al. in “Mol. Hum. Reprod.” Vol. 13, 2007, p. 11-20). A particular problem in cultivation of the cell aggregates is that until now no practicable, non-invasive method has been available for characterizing the cells contained in a cell aggregate.

The object to be achieved by the invention is to provide an improved method for receiving, in particular for separating biological cells from a stem cell culture, with which the disadvantages and limitations of the conventional technology are avoided. Another object is the provision of an improved substrate arrangement, with which in particular the disadvantages of the conventional combination of a carrier and a probe are overcome.

These objects are achieved by a method and a substrate arrangement with the features of the independent claims. Advantageous embodiments and applications of the invention are evident from the dependent claims.

According to a first aspect the invention is based on the general technical teaching, for providing a method for receiving biological cells from a cell culture, in which the cells move by their natural cell locomotion from a main substrate, on which the cell culture is arranged, onto one or more receiving substrates, on which a cultivation of the cells takes place. In contrast to the conventional cell transfer between a carrier, which forms a substrate for a cell culture, and a probe, which represents a transport tool for individual cells, according to the invention cell transfer takes place between substrates, both of which are set up for cultivation of biological cells.

With respect to the device, the stated object is achieved by a substrate arrangement with at least two substrates, which are positioned contiguous with one another so that the cells can be transferred by their natural cell locomotion from one of the substrates to the adjacent substrate. The substrates comprise a main substrate, on which a cell culture can be cultivated, and at least one receiving substrate, which is set up for colonization with cells from the cell culture of the main substrate and for cultivation of these cells. The main substrate and the at least one receiving substrate are separate components, which can be positioned contiguous with one another for cell transfer, and can be separated from one another after cell transfer.

An important advantage of the invention is that numerous disadvantages and limitations of the conventional technology are overcome. With the at least one receiving substrate, an individual cell can be collected, or a plurality of cells (e.g. at least 10, 100, 1000 or even a million or more cells) can be collected simultaneously, from the main substrate. The cultivation of the cells on the at least one receiving substrate makes it possible for the cells to be submitted to further processing, especially investigations or treatments, on the receiving substrate without further intermediate steps. For transferring the cells onto the at least one receiving substrate it is only necessary for this to be arranged to fit an edge of the main substrate. The transfer of the cells does not require any feedback-controlled manipulation of the substrates, nor any special measures for the adjustment of the main and receiving substrates relative to one another before or during cell transfer.

According to the invention, the at least one receiving substrate is set up for a cultivation of the cells that migrated out of the cell culture. The receiving substrate has a substrate surface whose size and material are selected so that an adherent cell culture comprising at least one cell or a plurality of cells (preferably at least 10 cells, e.g. some thousand cells) can be formed on the receiving substrate. The cells can be cultivated on the receiving substrate, i.e. the receiving substrate is suitable for growth and/or differentiation of the cells under adjustable, reproducible culture conditions. A surface modification (e.g. molecular deposition or micro- or nano-structurization) can be provided on the receiving substrate.

According to a preferred embodiment of the invention, cells are transferred from at least one three-dimensional cell aggregate, which is arranged on the main substrate, onto the at least one receiving substrate. The three-dimensional cell aggregate is formed from stem cells and optionally a mixture of cells of various types and preferably comprises an organoid body, as described for example by C. Kruse et al. in the aforementioned publications. The organoid body preferably has a cross-sectional dimension (e.g. thickness, diameter) in the range from 50 μm to 20 mm. The inventors found that the property of organoid bodies, in the adherent state, of forming a three-dimensional cell cluster, from the surface of which cells grow out uniformly, offers a substantial advantage for the invention. The organoid body forms a fixed cell culture on the main substrate, i.e. during the transfer of cells onto the at least one receiving substrate the position of the organoid body remains unchanged.

In contrast to the conventional technology, therefore no special requirements are imposed on mutual orientation or even movement of the main and receiving substrates. Exclusively the outward-migrating cells can be received selectively from the cell aggregate, without the cell aggregate being impaired. No microscopic observation or control is required. The method can advantageously be automated. Moreover, the long-lasting proliferation of organoid bodies is utilized to particular advantage. The growth of cells from organoid bodies can be kept stable over long cultivation times, e.g. in the range from 1 or 2 days to 2, 10, 30 or more weeks. Therefore the receiving of cells onto the at least one receiving substrate can also cover a corresponding period.

With respect to the device, the aforementioned object is therefore achieved in particular by a substrate arrangement with the main substrate and the at least one receiving substrate, wherein at least one three-dimensional cell aggregate, which is formed from stem cells and out of which cells migrate, is arranged on the main substrate.

Advantageously, it would therefore be possible according to the invention for cells that migrate out of the organoid body to be transferred exclusively onto the at least one receiving substrate, whereas other cells, which are a part of the organoid body, remain on the main substrate. A selective removal of the cells migrating out of the stem cell culture in the organoid body becomes possible. Special measures, for instance an adjustment of the contiguous substrates or a monitoring of cell transfer, are not necessary. The method of receiving cells from the cell culture forms in this case a method of separation of cells from organoid bodies, wherein advantageously the origin and the path of the cells and optionally their variation (e.g. differentiation, dedifferentiation) can be monitored and documented.

If the cells from the cell culture on the main substrate are moved onto several different receiving substrates, there may be advantages from the potential to form a plurality of daughter-cell cultures on the receiving substrates, and/or from the possibility of exposing the cells on the different receiving substrates to different culture conditions.

Therefore according to a preferred variant of the invention the substrate arrangement comprises the main substrate and a plurality of receiving substrates, which are formed to fit the main substrate and can be positioned to be contiguous with the latter. A receiving substrate is formed to fit the main substrate when the receiving substrate can be positioned at the main substrate and an edge of the main substrate forms a boundary line, and when the latter is overcome by the natural cell locomotion, the cells move onto the receiving substrate. Preferably it is provided that the cells move from the main substrate directly onto the receiving substrate. The receiving substrates form a set of interchangeable parts, with which the main substrate can be composed successively. With each additional receiving substrate, additional cells can be received from the cell aggregate. According to the invention, a modular system can thus be created, in which the first receiving substrate can be replaced by at least one further receiving substrate or in which the first receiving substrate is followed by at least one further receiving substrate.

According to an advantageous variant of the invention, it can be envisaged that the cells that are received by the different receiving substrates differ in their properties. For the specific receiving of cells with different properties in each case on different receiving substrates, it is possible to use different inherent properties of the cells and/or different properties of the substrate surfaces of the receiving substrates. When cells migrate out of the stem cell culture on the main substrate, these cells can comprise various cell types, e.g. stem cells, precursor cells or differentiated cells. The inventors found that the various cell types have different migration speeds. Thus, based on a starting time, at which the main substrate and the receiving substrate are positioned to be contiguous with one another, different cell types are transferred onto different receiving substrates at different times.

Advantageously, this makes possible non-invasive separation of cells from the stem cell culture, in particular from the organoid body, depending on their cell types. The inventors furthermore found that, depending on their types of differentiation, the cells can migrate onto different receiving substrates, the receiving substrates being provided with a modified substrate surface, onto which exclusively a particular differentiation type migrates. The inventors have found indications that, depending on previously unknown intrinsic properties, the cells can migrate or grow onto different receiving substrates, the receiving substrates being provided with a modified substrate surface, onto which particular cells then migrate or grow.

When the transfer of cells onto a plurality of receiving substrates is envisaged, according to a first variant of the invention these can be provided sequentially at the main substrate. For example, in a first step the main substrate with the cell culture, in particular the organoid body, and a first receiving substrate are provided for cell transfer of a first group of cells and/or a first cell type onto the first receiving substrate. Then the main substrate and the first receiving substrate are separated from one another. The further cultivation of the cells on the first receiving substrate takes place separately from the main substrate. Then the steps of cell transfer and separation are repeated with at least one further receiving substrate, wherein further cells, in particular further cell types migrate from the cell culture of the main substrate onto the at least one further receiving substrate.

With the sequential cell transfer onto different receiving substrates, various advantages can be achieved. First, the aforesaid separation according to cell types with different migration speeds can be achieved, by providing the receiving substrates in predetermined time segments at the main substrate. Furthermore, a plurality of daughter substrates can be colonized with cells from the cell culture. Through the successive combination of the main substrate with the receiving substrates it is possible for example in one cultivation device to react a main substrate with an organoid body successively between different receiving substrates, so that in each case colonization with cells from the organoid body is achieved.

When transferring the cells onto different receiving substrates, it can be envisaged according to another variant of the invention to provide all receiving substrates simultaneously at the main substrate. The receiving substrates can for example be arranged to be contiguous with different sections of the edge of the main substrate. This design is suitable in particular for the transfer of cells with identical migration speeds onto receiving substrates with substrate surfaces modified in various ways. According to another example the receiving substrates can be arranged at the main substrate such that cells first migrate across a receiving substrate directly adjacent to the main substrate, before they reach a receiving substrate at a greater distance from the main substrate. In this case cells with high migration speeds migrate in one time segment onto receiving substrates arranged at a distance, while simultaneously cells with a lower migration speed migrate onto receiving substrates at a small distance from the main substrate. The migration of the cells can be directed or accelerated by means of chemotaxis, galvanotaxis or other attractors.

If, according to another embodiment of the invention, the cells migrate out of the cell culture on the main substrate in at least two different directions onto the at least one receiving substrate, there may be advantages from increased effectiveness of cell uptake from the cell culture. The cells can for example migrate onto a single receiving substrate, which the main substrate surrounds at least partially, or onto different receiving substrates, which are arranged correspondingly in different directions of cell migration. A variant in which the cells move radially in all directions from the cell culture, in particular from the organoid body on the main substrate, onto the at least one receiving substrate, is especially preferred. The inventors found that the speed of the cells migrating out of the cell aggregate increases as the density decreases. Therefore, advantageously, uniform colonization of the at least one receiving substrate is achieved.

Advantageously there is considerable variability in the design of the at least one receiving substrate. The invention can therefore be implemented under various application conditions and for various tasks. According to a first variant, a substrate frame is formed by the at least one receiving substrate, which surrounds the main substrate. The substrate frame is especially suitable for receiving cells that migrate in all directions from the main substrate onto the at least one receiving substrate.

Generally a receiving substrate is called a substrate frame when the substrate surface, which is provided for receiving and cultivating the cells, surrounds a region that remains cell-free during cell transfer. For example, the substrate frame can have an inner opening, which forms the cell-free region and into which the main substrate can be inserted. Alternatively, for forming the substrate frame, the receiving substrate can be a substrate with a closed substrate surface, on which the main substrate can be placed for cell transfer, so that an outer region of the substrate remains free as a substrate surface for receiving and cultivating the cells. In this case the substrate arrangement according to the invention comprises a stack of a main substrate and at least one receiving substrate.

The substrate frame therefore generally has an internal edge, which is matched to the shape and size of the edge of the main substrate. If, for example, the main substrate generally has a polygonal shape, then the internal edge of the substrate frame is complementary to the polygonal shape. However, a circular shape of the main substrate and of the internal edge of the substrate frame is preferred. In this case the substrate frame is also called a substrate ring. The circular shape offers advantages for uniform transfer of the cells onto the receiving substrate radially in all directions.

According to another alternative, the substrate frame can be made up of several receiving substrates (substrate segments), which can be positioned on different sides contiguous with the main substrate. The substrate segments are components that can be separated from one another, which in the assembled state form the substrate frame. With the substrate segments, substrate surfaces modified in various ways can advantageously be provided for a cell transfer. The cells separated simultaneously from the cell culture on the main substrate can be submitted to different culture conditions on the substrate segments.

According to another advantageous embodiment of the invention, the receiving substrates can form a composite substrate. The main substrate and the composite substrate can be positioned relative to one another such that the main substrate is in each case contiguous with a receiving substrate of the composite substrate. Moreover, the main substrate and the composite substrate can move relative to one another. Thus, by mutual displacement of the composite substrate and the main substrate, a predetermined receiving substrate can be provided for receiving cells and/or this receiving substrate can be separated from the main substrate after receiving the cells.

A variant of the invention in which the composite substrate forms a flexible substrate strip, in whose surface the receiving substrates are formed, is especially preferred. A series of receiving substrates is provided along the length of the substrate strip. The substrate strip is arranged for a translational movement relative to a lateral edge of the main substrate. The lateral edge of the main substrate lies on the surface of the substrate strip. Thus, through translation of the substrate strip, in each case a predetermined receiving substrate can be provided at the main substrate. Advantageously, the substrate strip can be pulled past the main substrate by a substrate roll, in order to receive cells on the various receiving substrates.

When the displacement of the composite substrate relative to the main substrate is discontinuous, there are advantages for methods in which cells migrate alternately onto one of the receiving substrates and then the receiving substrate in question is separated from the main substrate. Alternatively the displacement of the composite substrate relative to the main substrate can be continuous. In this case the cells can be transferred continuously onto the composite substrate. Advantageously, large surfaces can thus be colonized with cells that have migrated out of a single cell culture, in particular a single organoid body. The mutual displacement of the composite substrate and main substrate preferably takes place at a speed that is adjusted to the speed of the natural cell locomotion. The speed is selected for example in the range from 1 μm/h to 400 μm/h.

Further advantages of the invention result from the possibility, after receiving the cells on the at least one receiving substrate, of carrying out further steps of treatment and/or investigation of the cells cultivated on the receiving substrate. Thus, immobilization of the cells on the receiving substrate can be provided, for example in order to prepare a test culture. Further growth and/or differentiation of the cells can take place on the receiving substrate. As the cells had migrated onto different receiving substrates out of a common cell culture, in particular out of a single organoid body, advantageously comparative investigations are possible. Moreover, after receiving the cells on the at least one receiving substrate, a further separation of differentiated and undifferentiated cells and/or an interaction of the cells with other biological cells can be provided. For example, the cells that were taken up with different receiving substrates and were there exposed to different culture conditions can be made to interact.

Further details and advantages of the invention are described below, referring to the appended drawings, which show:

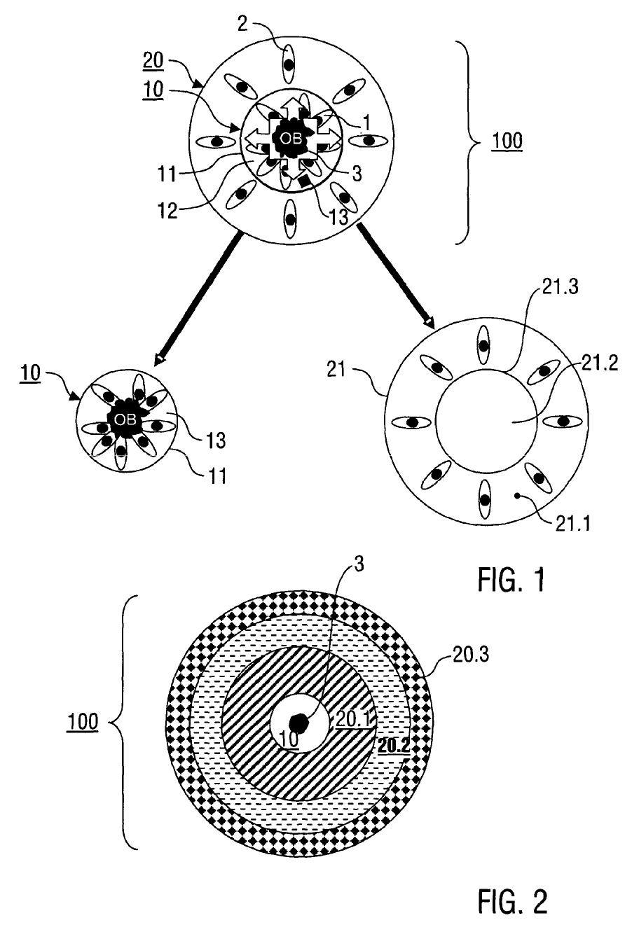

FIG. 1: a schematic illustration of a first embodiment of the invention with a cell locomotion onto a substrate ring;

FIG. 2: a top view of a substrate arrangement with a stack of main and receiving substrates;

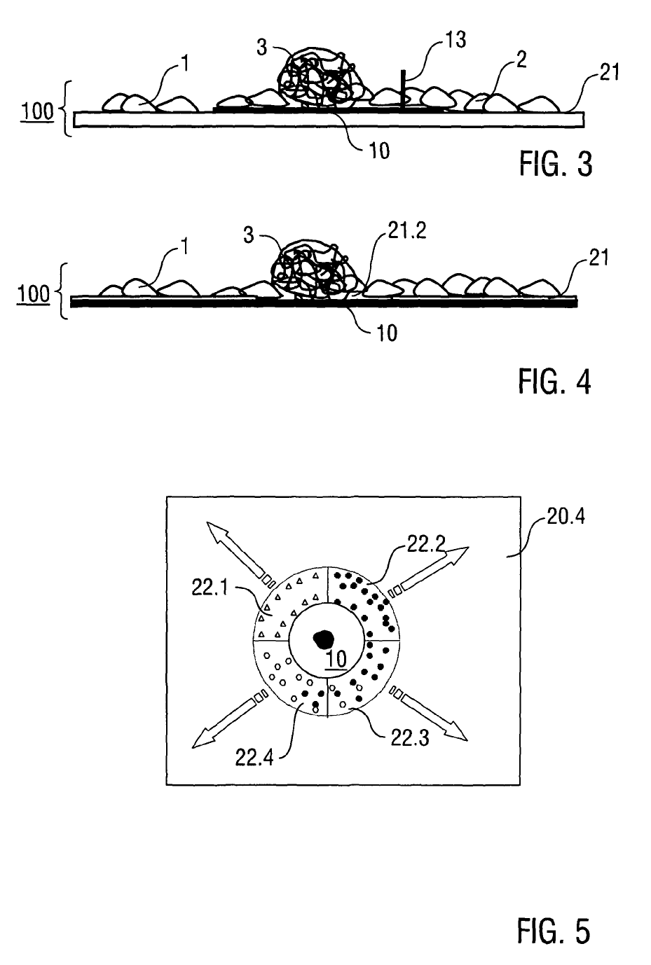

FIGS. 3 and 4: schematic side views of substrate arrangements according to the invention;

FIG. 5: another schematic illustration of the first embodiment of the invention with a cell locomotion onto substrate segments;

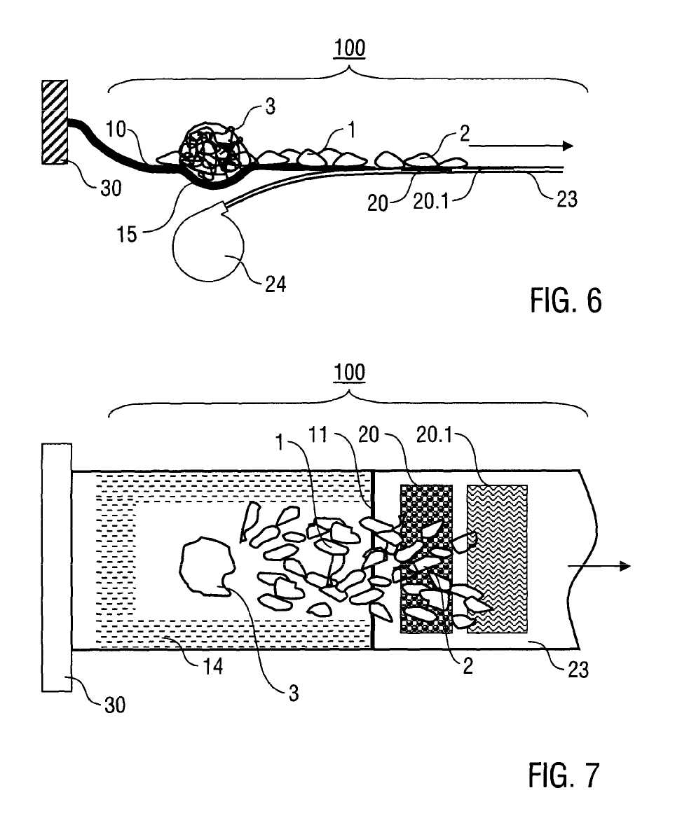

FIG. 6: a schematic side view of a second embodiment of the invention with a composite substrate; and

FIG. 7: a top view of the embodiment of the invention shown in FIG. 6.

Embodiments of the invention are explained in the following with exemplary reference made to the receiving of stem cells and/or differentiated cells from cell aggregates (organoid bodies), which are formed from glandular stem cells. The implementation of the invention is, however, not limited to the use of glandular stem cells, but is also possible with other, adult or embryonic stem cells, precursor cells and cell mixtures of human or animal origin. The cell aggregates can contain different cell types, i.e. in addition to the stem cells, also precursor cells and/or differentiated cells. Alternatively cells can be received from a tissue isolate (e.g. dermis, hair follicle). Details of the cultivation of stem cells or other cell types are not described here, as these are known per se from the standard methods of cell biology.

Here, “substrate” generally means a component that is suitable for use as a carrier for biological cells. The substrate has a substrate surface, on which the cells adhere and grow and can perform the natural cell locomotion. The substrate is of a two-dimensional form, and consists of a solid material, which can be rigid (e.g. disk, plate) or flexible (e.g. film). During the transfer of the cells the main and receiving substrates are joined together. The main and receiving substrates have for example level substrate surfaces, which in the joined state are arranged directly contiguous with one another or overlapping. The substrate arrangement according to the invention is in a cultivation device, which is not shown in the drawings. The cultivation device comprises e.g. a conventional culture vessel with a liquid culture medium.

FIG. 1 is a schematic top view of a first embodiment of the substrate arrangement 100 according to the invention with a main substrate 10 and a receiving substrate 20. The main substrate 10 comprises a plate in the form of a circular disk with a circular outer edge 11. The main substrate 10 consists e.g. of glass or plastic. A coating for providing predetermined culture conditions, e.g. a coating of laminin, polylysine, gelatin, fibronectin or other peptides, can be provided on the level substrate surface 12 of the main substrate 10. The diameter of the main substrate 10 is selected e.g. in the range from 0.5 mm to 2 cm. The thickness of the main substrate 10 is selected e.g. in the range from 10 μm to 2 mm. The main substrate 10 can be equipped with a schematically illustrated carrier element 13 (see FIG. 3). The carrier element 13 is a projection, with which a manipulation of the main substrate 10 using a mechanical tool is facilitated.

The receiving substrate 20 comprises a substrate ring 21 with a substrate surface 21.1, which is set up for a cultivation of biological cells. The substrate ring 21 has an inner opening 21.2, which is bounded by the internal edge 21.3 of the substrate ring 21. The shape and size of the internal edge 21.3 are selected to have the same shape and size as the outer edge 11 of the main substrate 10. The outside diameter of the substrate ring 21 is selected e.g. in the range from 5 mm to 5 cm. The main substrate 10 is a positive fit in the inner opening 21.2 of the substrate ring 21. The surfaces of the main substrate and of the receiving substrate are preferably in alignment. The main substrate 10 and the substrate ring 21 are separate components, which can be assembled for the cell transfer according to the invention.

FIG. 1 also provides a schematic illustration of the steps of the method according to the invention for receiving cells from a cell culture. In a first phase, the cell culture 3 is cultivated on the main substrate 10. The cell culture 3 comprises one or more organoid bodies containing stem cells. The cultivation of the cell culture 3 with formation of the organoid body on the main substrate 10 takes place while the main substrate 10 is still separate from the substrate ring 21 or is already inserted in the latter.

After the formation of the organoid body, cells 1, 2 migrate out of the cell culture 3 radially in all directions (see arrows). Owing to the natural cell locomotion, the cells 1, 2 migrate onto the substrate ring 21, on which further cultivation of the cells takes place.

After the cell transfer of cells 1, 2 onto the substrate ring 21, the main substrate 10 and the receiving substrate 20 (substrate ring 21) are separated from one another (see bottom part of FIG. 1). The time point of separation is selected depending on the actual application conditions of the invention. For example, separation can take place after a predetermined transfer time (e.g. 1 hour to 14 days) or after reaching a predetermined degree of colonization of the receiving substrate 20.

Then, the main substrate 10 can be combined with another receiving substrate 20 (not shown in FIG. 1), in order to transfer additional cells onto the further receiving substrate. Furthermore, the cell culture formed on the substrate ring 21 can be submitted to further steps of treatment and/or investigation, e.g. further differentiation.

FIG. 2 illustrates a modified variant of a substrate arrangement 100, in which several receiving substrates 20.1, 20.2, 20.3 are combined with a main substrate 10 as a substrate stack. The main substrate 10 with the cell culture 3 (organoid body) comprises a circular disk, which lies on the first receiving substrate 20.1. The first receiving substrate 20.1 also comprises a circular disk, which lies on the second receiving substrate 20.2, which in its turn lies on the third receiving substrate 20.3. As the receiving substrates 20.1, 20.2 and 20.3 have different diameters, with concentric arrangement a substrate ring is formed on each of the receiving substrates. For example, the first substrate ring, which surrounds the main substrate 10 on all sides, is formed by the portion of the first receiving substrate 20.1 not covered by the main substrate 10.

For efficient cell transfer, the thickness of the main substrate 10 is preferably selected to be less than 1 mm, in particular less than 250 μm. For this, the main substrate 10 consists, for example, of plastic film, e.g. of polyurethane. The thicknesses of the receiving substrates 20.1 and 20.2 also preferably have a thickness of less than 1 mm, in particular less than 250 μm. The contiguous edges of the main and receiving substrates can be beveled, to facilitate cell locomotion between the substrates.

For the receiving, according to the invention, of biological cells from the cell culture 3, the main substrate 10 with the cell culture 3 is placed on the stack of receiving substrates 20.1, 20.2 and 20.3. Owing to the natural cell locomotion, the cells migrating out of the cell culture 3 move radially in all directions onto the substrate rings. As the various cell types migrating out of the cell culture 3 may differ in their migration speeds, separation according to cell types can take place as a result of the cell transfer. Thus, after a predetermined time of cell transfer (e.g. 1 to 2 hours to 14 days) the quickest cells reach the outermost substrate ring 21.3, whereas the other cells are arranged on the inner substrate rings 21.1, 21.2. Then the main substrate 10 is separated from the receiving substrates 20.1, 20.2 and 20.3 and the receiving substrates are separated from one another. The main substrate 10 with the cell culture 3 is then placed on another substrate stack, to receive more cells from the cell culture 3. The receiving substrates 20.1, 20.2 and 20.3 are submitted to further cultivation steps.

The principle of formation of a substrate stack shown in FIG. 2 is not limited to the combination of the main substrate 10 with three receiving substrates, but correspondingly is also possible for example with a single receiving substrate or more than three receiving substrates. As an alternative to the arrangement shown of the main substrate with a small diameter on the receiving substrates with increasing diameters, the receiving substrates can comprise substrate rings with different inside diameters, which are placed on a main substrate. These two variants of formation of the substrate stack are illustrated schematically with the side views in FIGS. 3 and 4.

According to FIG. 3, the main substrate 10 lies, as described for FIG. 2, on a two-dimensional substrate. The receiving substrate 20 (substrate ring 21) is formed by its portion not covered by the main substrate 10. The cell culture 3, out of which cells 1, 2 migrate and are transferred onto the substrate ring 21, is arranged on the main substrate 10. After colonization of the substrate ring 21 by the cells 1, 2, the main substrate 10 with the cell culture 3 can be removed from the two-dimensional substrate and can be submitted to further processing steps. For separating the main and receiving substrates 10, 20, the main substrate 10 on the carrier element 13 is grasped with a mechanical tool (not shown).

FIG. 4 illustrates the inverse principle of formation of a substrate stack, in which the main substrate 10 forms a carrier for an annular receiving substrate 20 with an inner opening 21.2. The cell culture 3 rests motionless on the receiving substrate 10 in the inner opening 21.2 of the receiving substrate 20. The cells 1, 2 migrating out of the cell culture colonize the substrate surface of the receiving substrate 20, where a further cultivation of the cells 1, 2 also takes place.

FIG. 5 illustrates another variant of the invention, in which several substrate segments 22.1, 22.2 and 22.4, which surround the main substrate 10, are provided as receiving substrates 20. In the assembled state, the substrate segments 22.1 to 22.4 form a substrate ring that can be colonized by cells, as was described above with reference to FIG. 1. After the cell transfer, substrate segments 22.1 to 22.4 can be separated from one another (see arrows). After separation, different substrate segments can be combined with one another, to cause interaction of the cells on the substrate segments.

According to a variant of the invention, the separation can comprise a displacement of the substrate segments 22.1 to 22.4 on a common carrier, which forms another receiving substrate 20.4. In this case the cells can migrate from the substrate segments 22.1 to 22.4 onto the further receiving substrate 20.4 and can interact with one another on the latter.

FIG. 5 illustrates schematically that the receiving substrate 20 can have a biologically active coating. The biologically active coating can comprise e.g. fibronectin, another matrix molecule, a polymer or immobilizing signal molecules. The coating can be formed homogeneously (22.1), can have an areal density gradient (22.2) or can comprise at least one coating substance (22.3, 22.4). Different coating substances can optionally partially overlap in the transition between the substrate segments (22.3, 22.4). According to another alternative, patterns of the coating having detailed structures with typical dimensions in the μm or mm scale can be provided. By the biologically active coating of the substrate segments, different culture conditions are created on the different parts of the receiving substrate 20. For example the stem cells migrating out of the cell culture 3 can be differentiated differently on the substrate segments 22.1 to 22.4. After separation of the substrate segments and/or transfer onto the common receiving substrate 20.4, the differently differentiated cells can interact with one another. According to another variant, different receiving substrates can be colonized with cells from different cell cultures (not shown in FIG. 5). After cell transfer onto the receiving substrates, mutual interaction of the different cells can be envisaged.

FIGS. 6 and 7 show a second embodiment of the invention, in which the receiving substrates 20, 20.1, . . . form part of a substrate strip 23, in schematic side view and top view. The substrate arrangement 100 comprises the main substrate 10 for receiving the cell culture 3 and the substrate strip 23. The main substrate 10 is arranged fixed in a cultivation device and fastened e.g. to a wall 30 of a culture vessel (not shown). An adhesion-reducing coating 14 (e.g. of PTFE, alginate, polysaccharides) can be provided on the main substrate 10, to restrict the movement of cells to paths towards the edge 11. A trough 15 in the main substrate 10 serves for receiving the cell culture 3.

The substrate strip 23 comprises a flexible material, e.g. plastic, on whose surface the receiving substrates 20, 20.1 are formed. The receiving substrates 20, 20.1 comprise regions of the surface of the substrate strip 23 that are separate from one another. The substrate strip 23 has a thickness of e.g. 250 μm and a width of e.g. 10 mm. The receiving substrates 20, 20.1 are formed with different biologically active coatings, e.g. of laminin, fibronectin or other bioactive molecules, which are used for specific adhesion of cells.

The substrate strip 23 is arranged in a substrate roll 24 underneath the main substrate 10. By means of the substrate roll 24, the substrate strip 23 is pulled past the edge 11 of the main substrate 10 by deflection rollers (not shown), so that cells 1, 2 can migrate onto the receiving substrate 20, 20.1 that is contiguous in each case. The translational movement (see arrow) of the substrate strip 24 takes place for example at a speed in the range from 1 μm/h to 400 μm/h.

According to another variant, it can be provided that the strip is advanced discontinuously faster, to create sections with less cell colonization or without cell colonization. Advantageously the strip can then be cut at these sections, without damaging the other cells.

For the receiving, according to the invention, of biological cells from the cell culture 3, the latter is arranged on the main substrate 10. Owing to the natural cell locomotion, the cells 1, 2 migrating out of the cell culture 3 move over the edge 11 onto the adjoining receiving substrate 20. In order to receive further cells from the cell culture 3, the substrate strip 23 with the receiving substrates 20, 20.1, . . . is displaced relative to the edge 11 of the main substrate 10.

With the embodiment of the invention shown in FIGS. 6 and 7, different cell types can advantageously be separated from the cell culture 3 (comprising e.g. an organoid body or a tissue isolate) and can be received separately on surfaces. Further cultivation steps, treatments and/or investigations can then be carried out on the cells that have been separated from one another.

The features of the invention disclosed in the above description, the drawings and the claims may be of importance both individually and in combination for the practical application of the invention in its various embodiments.

1. A method for receiving biological cells from a cell culture, comprising the following steps:
cultivation of the cell culture on a main substrate,
cell transfer from the main substrate onto at least one receiving substrate, wherein cells move onto the at least one receiving substrate by natural cell locomotion, and
separation of the main substrate and of the at least one receiving substrate from one another,
wherein a cultivation of the cells is provided on the at least one receiving substrate.

2. The method according to claim 1, wherein the cell culture on the main substrate comprises a three-dimensional cell aggregate, which is formed from stem cells and out of which the cells migrate.

3. The method according to claim 1, wherein the cells move onto several different receiving substrates.

4. The method according to claim 3, wherein the cells, which move onto the different receiving substrates, in each case have different properties.

5. The method according to claim 4, wherein the cells, which move onto the different receiving substrates, have different migration speeds of the natural cell locomotion and/or different types of differentiation.

6. The method according to claim 1, wherein the receiving substrates for the cell transfer are provided sequentially on the main substrate.

7. The method according to claim 1, wherein the receiving substrates for the cell transfer are provided simultaneously on the main substrate.

8. The method according to claim 1, wherein the cells move in at least two different directions from the cell culture onto the at least one receiving substrate during the cell transfer.

9. The method according to claim 8, wherein the cells move radially in all directions from the cell culture onto the at least one receiving substrate.

10. The method according to claim 1, wherein the receiving substrates comprise substrate frames, which are arranged for cell transfer in such a way that the main substrate is surrounded by the substrate frames.

11. The method according to claim 1, wherein the receiving substrates comprise substrate segments, which are arranged to be contiguous with the main substrate for the cell transfer.

12. The method according to claim 1, wherein
the receiving substrates form a composite substrate, wherein
for the provision and/or receiving of the receiving substrates, the composite substrate and the main substrate are displaced relative to one another.

13. The method according to claim 12, wherein
the composite substrate comprises a flexible substrate strip, on whose surface a series of receiving substrates is formed, and
for the provision and/or receiving, a translation of the substrate strip relative to an edge of the main substrate is provided.

14. The method according to claim 12, wherein the mutual displacement of the composite substrate and of the main substrate takes place discontinuously.

15. The method according to claim 12, wherein the mutual displacement of the composite substrate and of the main substrate takes place continuously.

16. The method according to claim 15, wherein the displacement takes place with a speed in a range from 1 μm/h to 400 μm/h.

17. The method according to claim 1, comprising at least one of the following steps:
immobilization of the cells on the at least one receiving substrate,
investigation of the cells on the at least one receiving substrate ,
receiving of differentiated and undifferentiated cells by the at least one receiving substrate,
interaction of the cells on the at least one receiving substrate with other biological cells, and
interaction of the cells on one of the receiving substrates with the cells on another of the receiving substrates.

18. A substrate arrangement for providing biological cells of a cell culture, comprising:
a main substrate, on which the cell culture can be cultivated, and
at least one receiving substrate, which is adapted for receiving the cells, wherein
the main substrate and the at least one receiving substrate are formed so as to fit one another and can be positioned such that the cells can migrate by natural cell locomotion from the cell culture onto the at least one receiving substrate, and
the main substrate and the at least one receiving substrate can be separated from one another,
wherein the at least one receiving substrate has a substrate surface on which the cells can be cultivated.

19. The substrate arrangement according to claim 18, wherein several receiving substrates are provided, wherein the main substrate and the receiving substrates are formed so as to fit one another and can be positioned such that the cells can migrate by natural cell locomotion from the cell culture onto the at least one receiving substrate.

20. The substrate arrangement according to claim 19, wherein the at least one receiving substrate has an edge whose geometric shape is formed to be complementary to an outer edge of the main substrate.

21. The substrate arrangement according to claim 18, wherein the at least one receiving substrate forms a substrate frame that surrounds the main substrate.

22. The substrate arrangement according to claim 21, wherein each substrate frames comprises a substrate ring, which surrounds the main substrate.

23. The substrate arrangement according to claim 18, wherein the at least one receiving substrate comprises several substrate segments that can be separated from one another, which can be positioned on different sides so as to be contiguous with the main substrate.

24. The substrate arrangement according to claim 23, wherein
several receiving substrates form a composite substrate, on which an edge of the main substrate lies, wherein
the main substrate and the composite substrate can be displaced relative to one another.

25. The substrate arrangement according to claim 24, wherein
the composite substrate comprises a flexible substrate strip, on whose surface a series of receiving substrates are formed, wherein
the substrate strip is set up for translation relative to the edge of the main substrate.

26. The substrate arrangement according to claim 24, wherein the main substrate and the substrate strip are set up for relative movement with a speed in a range from 1 μm/h to 400 μm/h.

27. The substrate arrangement according to claim 18, wherein the main substrate and the at least one receiving substrate form a stack.

28. The substrate arrangement according to claim 18, which has at least one of the following properties:
at least one of the receiving substrates has an immobilizing surface, which is adapted for the immobilization of the cells,
at least one of the receiving substrates has a surface modification, by which the cultivation of the cells is promoted,
at least one of the receiving substrates carries additional biological cells.

29. The substrate arrangement according to claim 18, wherein the cell culture is arranged on the main substrate, and wherein the cell culture comprises a three-dimensional cell aggregate, which is formed from stem cells and out of which the cells migrate.
